(19) United States
(12) Patent Application Publication
(10) Pub. No.: US 2022/0126045 A1
(43) Pub. Date: Apr. 28, 2022

(54) ENDOTRACHEAL TUBE STABILIZATION DEVICE

(71) Applicant: The United States of America as Represented by the Secretary of the Navy, Silver Spring, MD (US)

(72) Inventors: Roy Dory, San Antonio, TX (US); Justin P Bequette, San Antonio, TX (US); Molly Marbut, Cibolo, TX (US)

(21) Appl. No.: 17/513,082

(22) Filed: Oct. 28, 2021

Related U.S. Application Data
(60) Provisional application No. 63/106,561, filed on Oct. 28, 2020.

(51) Int. Cl. A61M 16/04

(52) U.S. Cl. CPC A61M 16/0497; A61M 2209/088; A61M 2205/0238

(57) ABSTRACT

An endotracheal tube stabilization device comprising an endotracheal tube holder with an elongated generally tubular body and a bite block, a removable rotating wing plate on said endotracheal tube holder, and a strap assembly.

### CROSS-REFERENCE TO RELATED APPLICATIONS

This application claims the benefit to U.S. Provisional Application No. 63/106,561 filed Oct. 28, 2020, which is hereby incorporated by reference.

### FIELD OF THE INVENTION

This invention relates to a medical instrument, and specifically to an improved apparatus and method for stabilizing and securing an endotracheal tube to a patient.

### BACKGROUND OF THE INVENTION

Intubating a patient ensures that clinicians can provide oxygen, ventilation support, and administer medications to the affected casualty. During endotracheal intubation, an endotracheal (ET) tube is inserted through the oral cavity into the upper airway, securing the ability to mechanically ventilate the lungs of the casualty without airway obstruction. An endotracheal tube stabilization device (ETSD) is used to anchor and secure the endotracheal tube to the intubated patient. ETSD maintains endotracheal tube placement while minimizes the risks of comorbidities and extubation. In some designs, ETSD contains a bite block, which protects the endotracheal tube from damage or obstruction caused by the patient's mastication force.

Currently, there are a number of ways and products on the market for stabilizing an endotracheal tube (U.S. Pat. Nos. 4,270,529, 4,867,154, 490,504, 5,803,079, 5,996,581, 8,096,300, and US Patent Pub. Nos 20020092536 and 2017197049). Existing stabilizing devices typically consist of a holder assembly that is positioned over the patient's open mouth, which rests on the patient's cheeks, outer lips, and/or teeth. The holder clamps to the endotracheal tube, securing the depth and position of the endotracheal tube. The endotracheal tube holder (i.e. ETSD) is then secured to the patient using adhesive tape, fabric tape, or adjustable straps. Bite blocks are often used in conjunction with the endotracheal tube holder, which may be a separate device, or integrated into the ETSD. The bite block is typically made of rigid plastic, which surrounds the endotracheal tube within the oral cavity. Bite block prevents the patient from biting into the endotracheal tube, which can deform and damage the endotracheal tube.

However, conventional endotracheal tube fixation/stabilizing devices (ETSD) are not well suited for patients with maxillofacial burns or other facial injuries. The problems of stabilizing an endotracheal tube with adhesive tape have been well documented. When patients are ventilated by mechanical ventilator, warm, humidified gasses are used to avoid airway drying, and prevents the body from giving up moisture. The inhaled gas warms the endotracheal tube and reduces the adhesiveness and securing ability of the tape. This increases the possibility of inadvertent extubation and dislodgement of the tube, creating a potentially life-threatening situation. Stabilizing devices using adhesive tape are especially unsuitable for burn patients. The adhesive can further damage injured facial tissue, and does not adhere well due to burn creams, wound exudate and oral secretions, which greatly increases the chances of inadvertent dislodgement.

Strap- or harness-based fixation devices alleviate this issue by wrapping circumferentially around the head. However, the straps exert pressure against facial tissue, often rubbing against and irritating the corners of the mouth and cheeks of the patient. Many straps/harnesses systems have large contact areas that make visual examination of the skin beneath them difficult, and can cause infection or necrosis if the underlying tissue is already compromised. To resolve these issues, some clinicians, such as the US Army Institute of Surgical Research Burn Intensive Care Unit, uses twill tape to tie the endotracheal tube in place. Twill straps are frequently replaced in response to fluctuating facial swelling and to prevent tissue damage and infections where prolonged contact is made. However, the process of strap replacement or adjustment is both tedious and time consuming. A typical session can take two respiratory therapists up to 20 minutes, during which the endotracheal tube is not fully secured. Therefore, there is a critical need in the field, for a better designed endotracheal tube stabilizing device that minimizes skin and tissue damage while permitting easy application and the removal of fasteners for routine oral/wound care.

### BRIEF DESCRIPTION OF THE DRAWINGS

FIG. 1 is a perspective view of an endotracheal tube stabilizing device of this invention.

FIG. 2 is a top plan view of the wing of the endotracheal tube stabilizing device of this invention.

FIG. 3 is a side view of endotracheal tube stabilizing device of this invention.

FIG. 4 is a perspective view of endotracheal tube stabilizing device with full strap assembly.

FIG. 5A is perspective view of the endotracheal tube holder.

FIG. 5B is the side view of the endotracheal tube holder.

FIG. 6A-C shows stress test results. An external load of 1500 N was exerted on the bite block and a von Mises pressure map was created. Three materials were simulated: A. ABS. B. Polypropylene Copolymer and C. Low-Medium Density Polyethylene.

FIG. 7 shows pressure-mapping results from the corner of the mouth on the SynDaver manikin. Each box plot shows the average amount of pressure applied to the mouth by the three test devices with the standard deviations. The graph depicts which devices applied the most (BICU) and least (Tube Tamer) pressure.

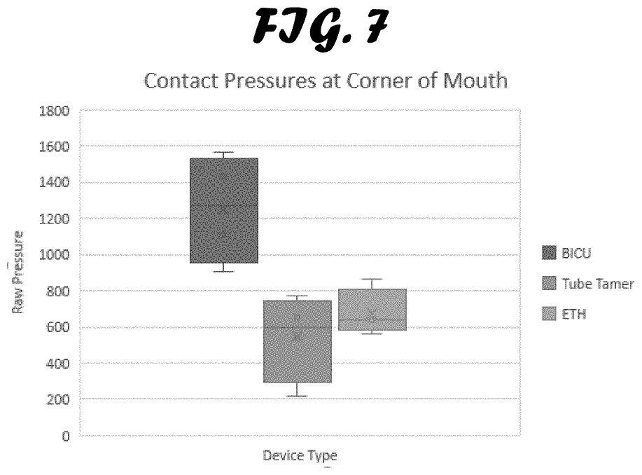

FIG. 8 shows pressure-mapping results from the Ear on the SynDaver manikin. Tube Tamer measurements came from the corner of the jaw (the device does not cover the ear). Each box plot shows the average amount of pressure applied by the three test devices, with standard deviations. The graph depicts which devices applied the most (BICU) and least (Tube Tamer and ETH) pressure.

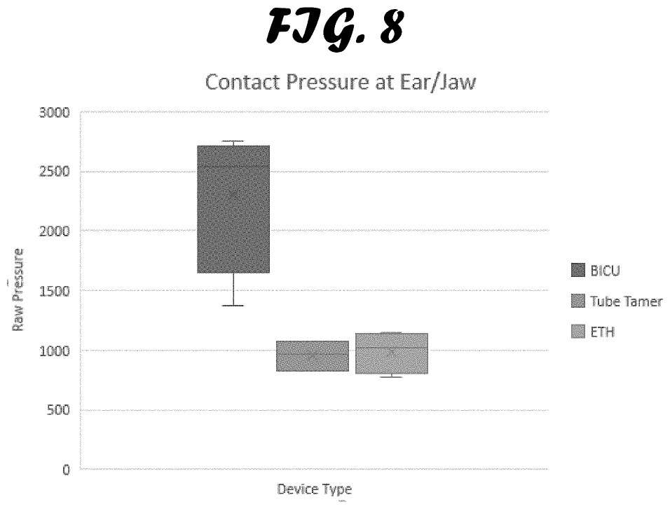

FIG. 9 shows extubation forces of each test device. Each box represents the average extubation forces of the three devices as well as the standard deviations.

### SUMMARY OF THE INVENTION

An objective of this invention is an endotracheal tube stabilization device (ETSD), which minimizes compromised tissue damage from the use of ETSD by reducing the localized pressure on the skin and face.

Another objective of this invention is an ETSD that incorporates a burn-compatible material to avoid irritating burned facial tissue.

Yet another objective of this invention is an ETSD that allows easy replacement without time-consuming adjustment by the care providers.

### DETAILED DESCRIPTION OF THE INVENTION

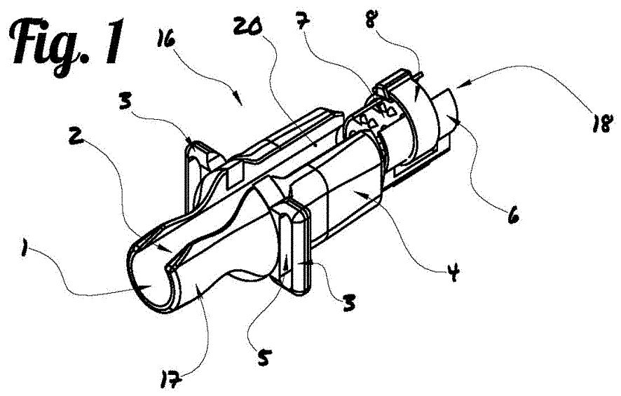

FIGS. 1-5 illustrate a prototype endotracheal tube stabilization device (ETSD) according to the present invention. The ETSD comprises an endotracheal tube holder (ETH) (16) with a bite block (4); a removable rotating wing plate (3) around the endotracheal tube holder; and a strap assembly (22) that secures the ETSD to a patient's head via the wing plate.

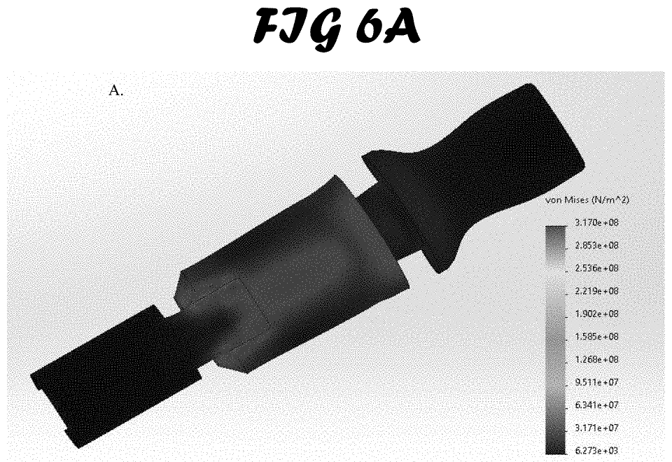

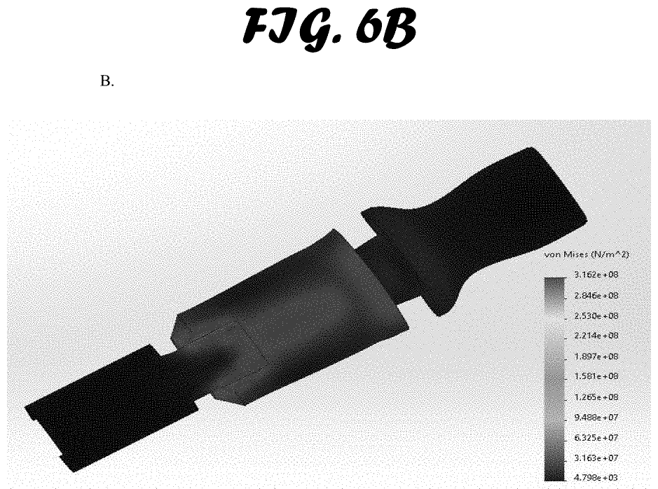

With reference to FIGS. 5A and B, the endotracheal tube holder (ETH) (16) has a substantially straight elongated tubular body that is adapted to removably receiving an endotracheal tube. The ETH (16) is made of semi-rigid materials, such as plastic or silicone. The body of the ETH comprises a proximal portion (17), an intermediate bite block (4) and a distal portion (6). The proximal portion (17) is adapted to be inserted into the oral cavity of a patient. The intermediate bite block (4) partially extends into the oral cavity, past the patient's incisors. The distal portion (6) extends outside of the patient's mouth. The body of ETH (16) have an axial bore (1) extending therethrough for removably receiving and passing an endotracheal tube therethrough, which has predetermined diameters. A slot (20) is made along the length of the axial bore (1) and is sized and adapted to allow the endotracheal tube holder (16) to be fitted onto or removed from an endotracheal tube by the side while the endotracheal tube has been inserted into the patient. The slot at the proximal end (2) of the ETH (16) is wider, which helps to guide the ETH onto the endotracheal tube during operation.

The intermediate bite block (4) of the ETH (16) may be an integrated portion of the ETH or a separate bite block fitted onto the intermediate portion of the ETH (i.e. between the proximal portion and the distal portion). In one embodiment, the intermediate bite block (4) has a generally square outer shape, and partially extends into the oral cavity, past the patient's incisors. The bite block's square outer shape helps to distribute the bite pressure between multiple teeth and minimizes potential dental damage to the patient. In another embodiment, a softer material may also be used to coat the outside surface of the bite block to further improve patient comfort.

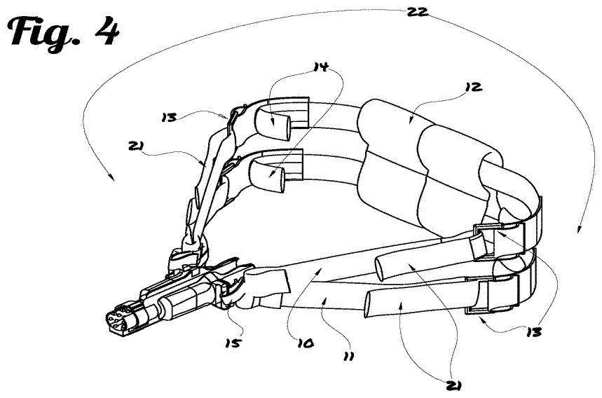

The axial bore of the distal portion (18) of ETH (16) may be semicircular, which surrounds and supports the endotracheal tube in operation. A grip pattern (7) lines the inner wall of axial bore of the distal portion (18), while the outside of the distal portion (6) of the ETH contains an adjustable fastener (8, FIG. 1), such as a cable tie. Upon positioning the ETH (16) onto the endotracheal tube, the adjustable fastener (8) is tightened. The ETH (16) engages the endotracheal tube by the grip pattern (7) inside the distal portion (6) of the ETH, locking the endotracheal tube in place in relation to the ETH (16). The adjustable fastener (8) and the grip pattern (7) together enable the ETH (16) of the present invention to accommodate and secure endotracheal tubes of various sizes. The bottom outside surface of distal portion (6) may further comprise a raised area (9, FIG. 3), which provides a place for taping an oral gastric tube.

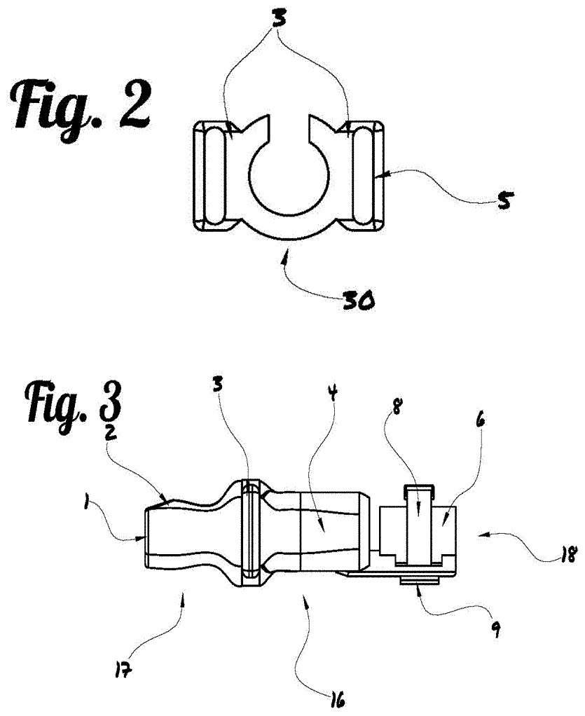

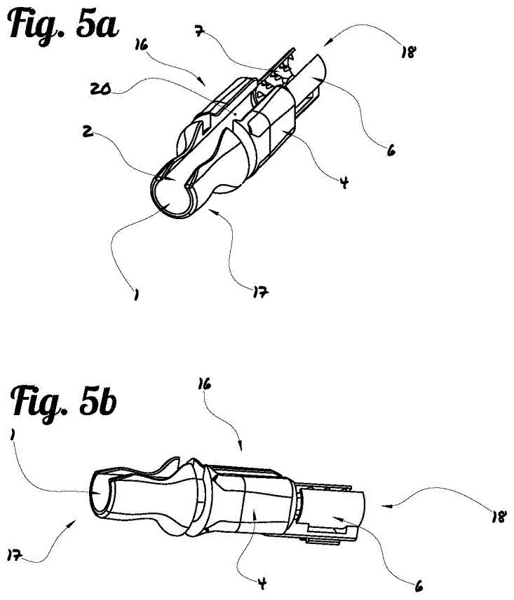

With reference to FIG. 2, the ETSD of this invention further comprises a removable rotating wing plate (3). The rotating wing plate (3) has two wings and center hole, which is adapted to fit onto the ETH (16) between the proximal portion (17) and the intermediate bite block (4). In an alternative design, the wing plate may comprise additional wings. Each wing of the wing plate (3) have a slot on its periphery (5), which engages a fabric strap assembly (see FIG. 4) that wraps around the patient's head. Once fitted, the wing plate (3) may rotate independently about the axis of the ET tube, to hold the ET tube in place and allows adjustment or easy replacement of the strap assembly.

In reference to FIG. 4, the ETSD may further include a strap assembly (22), which secure the EDST to the patient's head. The strap assembly (22) can be configured to use a single strap (10) or dual straps (10 and 11). In the dual straps configuration, a cushion (12) serves as a soft barrier between the patient's head and hospital bedding, decreasing the possibility of skin breakdown, and maintaining the proper distance between straps (10) and (11) behind the patients head. The strap assembly (22) is adjustable. Fastener such buckles (13) are used to assure an appropriate fit to patient's head while securing ETSD in place regardless of the patient's head size. Once pass through the cushion (14), the ends of the straps extends behind the buckles (13) to prevent further skin/tissue damage to the patient's head by properly distributing pressure against the patients head. Lastly, the portion of the strap(s) that connects to the wings (15) can be sewn or attached to the wing using a reusable fastener. They are shaped in such a way that pressure from the straps is diffused over the corners of the patient's mouth. In one embodiment, the fabric strap assembly in contact with the patient's skin incorporates an inner lining with a burn-compatible material, to decrease or eliminate further damage to injured skin tissue. Wound dressing may be used includes but not limited to nonadherent films, fine mesh gauze, foams, alginates, hydrocolloids, and hydrogels, and may further also include topical antimicrobials agent depending on the specific needs of the burn.

To apply the ETSD to an intubated patient, the proximal portion (17) of the ETH (16), is oriented toward the patient's oral cavity and guided onto the ET tube by pressing the ET tube through the slot (20) beginning from its wider section (2) and into the axial bore (1). The portion of the ETSD straps (15) that connect to the wing plate slots (5) are either sewn into place or secured using hook and loop fasteners. In the latter case, one end of the ETSD straps (15) are routed through the slot (5) of the wing plate (3), doubled back, and secured onto itself via a hook and loop fastener. The strap assembly is wrapped around the back of the patient's head and the unsecure end is routed through the second wing slot (5) on the other side of the ET, and secured in the same manner as previously described. The strap cushion (12) is centered on the back of the head. Two adjustment ends of the strap (21), four if dual straps are used, are equally pulled taut, which slides the ETSD down the ET tube, and positions the bite block (4) between the patient's incisors. The wing plate (3) is then rotated 90 degrees, either clockwise or counter clockwise around the ET tube, allowing the rotating wing plate (3) to close the slot (20) on the proximal portion (17) and hold the ET tube in place. The adjustable fastener (8) is tightened, and which engages the endotracheal tube with the grip pattern (7) within the distal portion (6) of the ETH (16), locking the endotracheal tube in place in relation to the endotracheal tube holder.

### Example 1: Testing of the Prototype ETSD

### Equipment

SynDaver™ Synthetic Airway Trainer (SynDaver™ Labs, Tampa, Fla.) is used to test the prototype ETSD. Each component of the airway trainer is designed to mimic the geometry as well as the physical properties of their respective tissues, including the fiber content; modulus in tension, compression, and shear; and coefficients of friction (Sakezles 2009). The airway trainer also features a hard and soft palate, tongue, uvula, epiglottis and vocal cords. This unique synthetic cadaver system allows for a thorough evaluation of the placement of an ET tube/device. When compared to a whole-body trainer, this model allows the user to observe where the tube was placed (trachea or esophagus) and verify through the tubes that protrude out of the upper torso. This model also provides a platform for repeatability studies that would not be safely feasible for extended durations with human subjects.

I-SCAN® Pressure Measurement System (TEKSCAN®, South Boston, Mass.). The I-SCAN® is a force and pressure measurement system, which displays and records dynamic and static interface pressure distribution data. The system includes Windows-based software, scanning electronics, and pressure sensors. The scanning electronics rapidly record pressure data from an array of independent sensing elements contained within each sensor. A Model 5101 sensor was used to measure and map the pressure exerted by the various ETSDs. The specific sensors used in this testing were rated for 50 psi. The Model 5101 has a 4.40″×4.40″ sensing matrix, which contains 1,936 individual sensing elements, to provide a spatial resolution of 100 elements per square inch. Data from the sensors was collected at a rate of 1 Hz and analyzed to determine the pressure distributions and the average contact pressures exerted on the sensing matrices.

FORMLABS® Form 2: Affordable Desktop SLA 3D Printer (FORMLABS®, Somerville, Mass.). The FORMLABS® Form 2 machine is a desktop 3D printer that uses stereolithography (SLA) to cure solid isotropic parts from a liquid photopolymer resin. The printer includes a resin tank, a build platform, a finish kit, and the PREFORM® Windows-compatible software. Additionally, various proprietary resins were obtained from Formlabs to print prototypes of the novel ETH for preliminary evaluation. “Durable” resin, in particular, was picked as a suitable resin for prototyping due to its ability to withstand considerable compression and tensional loads when compared to other Formlabs resins.

PCE INSTRUMENTS®-Digital Force Gauge N 200 (PCE INSTRUMENTS®, Jupiter, Fla.). The PCE INSTRUMENTS® digital force gauge is a digital force meter that allows for precise compression and tensile force measurements with a resolution of 0.1 N. This device includes multiple compression and tensile measuring adapters, an extension bar, power supply, USB data cable, and data analysis software. The digital gauge was secured in place over a fully intubated SynDaver Airway Trainer and was manually tested with each ET tube device. The data recorded was transferred from the force gauge to a laptop through the USB cable for analysis.

### ET Tube Stabilization Techniques Teste

The ET tube holder methods tested were the BICU Twill Tie Method (US Army Institute of Surgical Research Burn Intensive Care Unit, San Antonio, Tex.), the ErgoMed Tube Tamer model B7013 (Tube Securing Devices, ErgoMed Inc, San Antonio, Tex.), and the novel Endotracheal Tube Holder (ETH; Version 6.7; Naval Medical Research Unit, San Antonio, Tex.).

The BICU Twill Tie Method. The BICU method involves drilling holes into a commercially available bite block (Southmedic, Inc., Ontario, Calif.) and wrapping non-adhesive twill tie around the face of the patient in order to fix the ET tube in place. The method requires the modification and combination of multiple products and the application can vary depending on the provider who applies it. The provider must tie the twill tape tight in order to maintain stabilization of the ET tube. Self-adhesive silicone gel pads marketed for scar treatment (Cica-Care, Smith & Nephew, Watford, UK) are now added under the twill tape at the corners of the mouth. This is intended to reduce the pressure and cutting effect at the corners of the mouth, but was still not an ideal configuration as the pads were only secured in place by the pressure of the twill tape on the face in that area. The gel pads are prone to dislodging, especially once burn cream is applied to the area. One RT stated that they have tried to staple the pads to the twill tape, but that it was difficult to achieve proper positioning, could damage the skin if not done correctly and required extra equipment (a stapler and staples). To evaluate the most current BICU method, testing was performed with the gel pads in place.

The ErgoMed, Inc. Tube Tamer. The Tube Tamer is an ET tube fastener device that was created to address the ET tube stabilization issue when caring for intubated patients. This device incorporates a simple tape wrap and pad.

The Inventive Endotracheal Tube Holder. The prototype endotracheal tube stabilization device of this invention incorporates an endotracheal tube holder with an integrated bite block. The device is comprised of a cylindrical channel in which the ET tube is inserted. An opening to the channel allows the device to be fitted onto the endotracheal tube from the side, after the tube has been inserted into the patient. The front of the channel opening widens to help guide the device onto the endotracheal tube during application. Two rotating wings extend on either side of the device, proximal to the bite block section. The wings on the device have two slots (5) at their periphery, which interface with a fabric strap assembly that wraps around the patient's head. Additionally, this design allows the wings to be rotated independently about the axis of the ET tube, securing it in place. Behind the wings, the bite block section partially extends into the oral cavity, past the incisors. The bite block section has a square outer shape in order to distribute the bite pressure between multiple teeth to minimize potential dental damage when the block is bitten by the patient. Additionally, the device consists of a tube grip section, which is comprised of a semicircular barrel channel that surrounds the endotracheal tube. A grip pattern lines the inner wall while the outside contains an adjustable cable tie fastener. Upon inserting the device onto the endotracheal tube, the adjustable cable tie is tightened, engaging the grip within this section and locking the tube in place in relation to the device. The cable tie allows the use of multiple endotracheal tube sizes. The raised feature on the bottom of the device provides an area to tape an oral gastric tube. The fabric strap assembly is made up of a large pad that rests against the back of the patient's head and a set of 4 straps that can be independently loosened or tightened as necessary. These straps are outfitted with hook-and-loop fasteners to allow for easy adjustments by the respiratory therapists.

### Test Procedures

Bite Force Simulation Testing. Material types used to 3D print the ETH device prototypes are not intended to be the final material used for manufacturing. Therefore, material properties of three plastics commonly used in medical devices were chosen to be simulated: ABS, polypropylene and polyethylene (Kucklick 2013). Other suitable materials can also be used for the manufacturing of the inventive ETSD. As previously stated, casualties with maxillofacial burns are very often intubated to protect their airway. The BICU stated that, when intubated, their protocol is to sedate their patient. Bite forces of sedated patients differ from that of fully conscious subjects. Past research has shown that patient bite force increases with the administration of sedatives (Matsuura 2017). Variables such as the medication(s) selected and the dose administered are a factor. The highest bite force seen in the Matsuura review of intravenously sedated dental patients (1500 N) was used as references to simulate teeth compression on the bite block. This data was primarily used in the design and development of the bite block cross-sectional form factor. Performance of each prototype design iteration of the ETH bite block section were tested under static bite force loads of 1500 N within the force simulation component of the Solidworks CAD software (SimulationXpress, Solidworks, Dassault Systemes, Waltham, Mass.). Material property values for the bite block were selected to represent materials that may be chosen for the final device. Bite forces were then applied to the model and the resulting stress measurements were evaluated for future iteration design changes.

Test Platform Overview. The test platform system was designed to fit and secure the SynDaver Airway Trainer in place. The frame consists of a rigid plastic board which the SynDaver was placed on to take measurements. Holes were drilled into the board in order to install a strap system which fixed the head and shoulders of the SynDaver manikin in place. Rolled towels were placed under the neck to position the head in a more appropriate anatomical position, raising the chin away from the chest. This simulated the position of an intubated Burn ICU patient resting in a hospital bed.

The order in which each device was tested was as follows: the device was applied and secured to the SynDaver and ET tube, pressure mapping was performed at the corners of the mouth and ears, and an extubation force measurement was taken. This procedure was completed four times per method before moving on to the next device. The same person ran all four tests on all devices to control for inter-person variability as this was not intended to be a factor in the assessment at this time. Specific steps for each procedure are detailed below.

Device Application. Effort was taken to ensure devices were applied and tightened to clinically relevant levels for each specific device. The BICU device was applied and tightened by a trained BICU RT with experience in applying and assessing proper tightness and placement on a BICU patient. The Tube Tamer was applied per manufacturer instructions for use, by a researcher with experience caring for intubated patients. The instructions did not indicate a specific tightness, so it was tightened to what was estimated to be used in a clinical environment. The ETH was also applied by a researcher, and tightened to the level required to properly function as designed, aligning the bite block portion of the device with the Syndaver top incisors.

Pressure Map Testing. Once the manikin was secured to the board, two model 5101 pressure pads (50 psi) were placed in the desired locations (one folded into the right corner of the mouth and the other covering the left ear or left jaw for the Tube Tamer) on the manikin and fixed in place using medical tape. The sensor on the corner of the mouth was attached to the I-SCAN® receiver, connecting the pad to the I-SCAN® software. Once the pads were secured in place and the software was ready to collect data, the manikin was intubated with an ET tube and the fixation method was applied to the manikin to keep the tube in place. A real-time pressure map was then recorded to capture the pressure applied to the sensor. A rectangular selection area was placed on the pressure map in the first region of interest, the corner of the mouth. Once the recording was saved, the receiver was then switched to the second pressure sensor covering the ear. A real-time pressure map was again recorded and a region of interest box representing the location of the ear was created. Once the pressure on both regions was recorded and saved, the extubation force testing described below was performed on the same fixation method. The BICU twill tie method was the first to be tested, as this method was hypothesized to create the largest amount of localized pressure on the regions of interest (corner of the mouth and the ear). The pads were set to read relative pressure. The pressure pads used during this testing are rated for 50 psi, which is much higher than the pressure applied by a typical ETSD. Because of this, the sensitivity of the pads had to be set to a very high level. The first trial on the BICU method was used to set the sensitivity level of the pressure sensor to ensure a full range of pressures could be recorded and the sensitivity level was then documented for use in the remainder of the trials.

Extubation Force Testing. For extubation force testing, a hole was drilled in the distal end of the ET tube. Twill tape was then routed through the hole and the ends tied, creating a loop. This allowed the ET tube to be pulled from the center of the cross section of the tube.

The twill tape that was looped through the ET tube was secured to the force gauge and the device was placed in very slight tension perpendicular to the ET tube. Based on previous studies, a two cm movement was classified as “extubation” (Wagner, 2014). The ET tube contained markings at 1 cm increments which were used to gauge movement. The ET tube depth at the level of the manikin's incisors was noted. Recording started when pulling on the force gauge commenced and the fore gauge was pulled away from the manikin at a steady rate with constant force. When the tube had moved out of the mouth by two cm relative to the starting measurement, the extubation force recording was stopped. Once the extubation force data had been saved for that trial, the device was removed from the manikin and another round of pressure testing as described previously was performed. This combined procedure was performed four times for each device.

### Results and Discussion

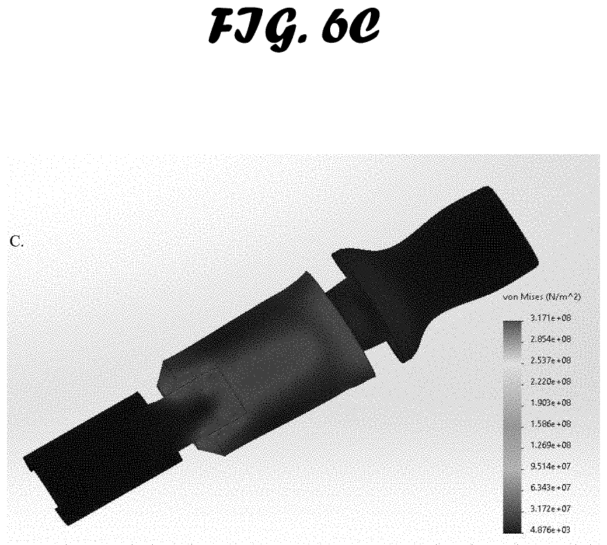

Bite Force Simulation Testing. Bite force simulation testing was limited to the ETH, and not used to compare the three methods tested in this study (FIG. 6A-C). Three common materials used in injection molded medical devices were simulated: ABS, polypropylene and polyethylene. Von Mises load stress testing was performed to examine each material's ability to withstand bending caused by a patient's bite. The results were used to validate the integrity of the bite block cross-sectional shape during iterative prototyping.

The results for all three material simulations with the prototype design were similar. The weakest point in the design was shown to be the surface perpendicular to the bite force applied. It was shown to absorb the force by flexing a very small amount. No materials experienced stresses high enough to cause breakage. It is also important to note that bending of the surface in question is possible due to a 7 mm open slot on the opposite surface of the bite block. When a force is applied, the slot closes and further bending is not possible unless the load is increased far above that seen in the bite of a patient.

Pressure Map Testing. Skin-to-device localized pressure map testing compared raw data scores between the three methods. Results were reported in the I-SCAN® software as a visual pressure map and raw data tables. Testing confirmed the initial hypothesis, which predicted that the BICU method would have the highest pressures at the two regions of interest, the corners of the mouth and ear.

The average pressure the BICU method applied at the corner of the mouth was 1253±301, while the pressures in the same location for the ETH and Tube Tamer were 677±131 and 546±241, respectively. Note, these raw pressure measurements are relative, not calibrated to a pressure standard, and are therefore dimensionless. While the cutting effect on the corners of the mouth was clinically found to decrease with the use of the silicone pads, contact pressures in that area remained higher than that of both the Tube Tamer and the ETH. The Tube Tamer device was found to have the lowest average pressure, 546±241, at the mouth corners. This was believed to be due to the method of application, wrapping of adhesive tape up the distal end of the ET tube, forcing the tape away from the mouth.

The Tube Tamer did not apply any pressure in the ear region, as the strap was routed around the neck with no contact at the ear. During the pressure testing on the Tube Tamer, the “ear” pressure was instead measured at the spot of highest pressure on the corner of the jaw. The average pressure placed on the corner of the jaw, 957±138, was comparable to the pressure the ETH applied to the ear, 988±184. The contact pressure of the BICU method at the ear, 2303±629, was the highest. While the Tube Tamer kept pressure at the corners of the mouth and ears to a minimum, addressing one of the BICU complaints, it was at the expense of extubation force, which is described below.

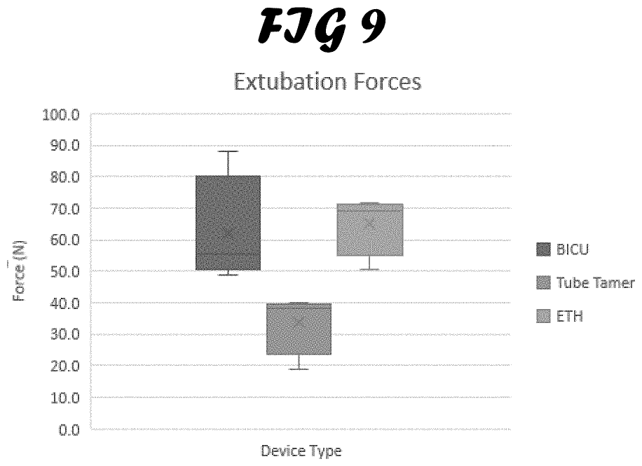

Extubation Force Testing. The extubation force testing was an important aspect of the development of the ETH prototype (FIG. 9). The Tube Tamer had the lowest average extubation force at 33.9±10.0 N. The BICU twill tape method averaged 62.0±17.6 N of extubation force and 65.2±9.7 N for the ETH. As noted previously, the patients in the BICU are often lightly sedated, increasing the possibility of extubation. As hypothesized, the Tube Tamer, while having lower device-to-face pressures than the other two methods, was also extubated with less force. This, and the lack of a bite block in the Tube Tamer design, would potentially present issues for the lightly sedated burn patients prone to self-extubation and biting of the ET tube. The ETH extubation forces were comparable to the BICU method, which provides confidence that the design is capable of preventing extubation in a similar manner as the BICU method while reducing the localized facial contact pressure. It is also important to note that informal extubation force testing by BICU staff, manually pulling on the tube while the ETH was secured to the SynDaver, received positive feedback. The RTs stated that the ETH felt secure enough to be effective, based on their clinical experience. Additionally, previous studies have shown that perpendicular extubation forces demonstrated by other fixation methods average from 31±7.7 N to 209±0.0 N for a 2 cm tube displacement (Wagner, 2014).

The extubation force testing on the ETH was performed by gripping the device itself as opposed to the distal portion of the tube like the other two devices. The goal was to test the ability of the devices to prevent extubation. The ETH prototype is 3D printed, therefore, not made of a material representing its final form and the 3D print material does not grip the ET tube well. However, extubation forces were still able to be measured by pulling from the body of the device. It should also be noted that the sample size obtained during this testing (N=4) was not large enough to make any statistically significant claims of performance. Despite this, the research team believes that the prototype performed well enough to establish confidence as a proof-of-concept. The ability of the ETH to decrease the pressure applied to the ears and mouth compared to the BICU technique in preliminary studies while successfully securing an ET tube achieved the goals of this project.

As the results shown, the inventive ETSD was able to decrease the pressure applied to the ears and mouth of the patient, effectively present extubation and allow easy adjustment of the strap assemblies.

### REFERENCES

Davis, C. “Endotracheal Tube Fixation to the Maxilla in Patients with Facial Burns” Plastic and Reconstructive Surgery, 113, no. 3 (2004): 982-84.
Johnson, B. W. et al. “Combat-related Facial Burns: Analysis of Strategic Pitfalls” Journal of Oral and Maxillofacial Surgery, 73, no. 1 (2015): 106-111.
Kucklick, T. R. “The Medical Device R&D Handbook.” Second Edition (2013). United Kingdom: CRC Press.
Matsuura, N. “Muscle power during intravenous sedation.” Japanese Dental Science Review, 53, (2017): 125-133.
Mitchener, T. A & Canham-Chervak, M. “Oral-Maxillofacial Injury Surveillance in the Department of Defense, 1996-2005” American Journal of Preventative Medicine, 38, no. 1 (2010): S86-S93.
Personal communications. Burn Intensive Care Unit, Institute of Surgical Research, Brooke Army Medical Center, San Antonio Tex., 2015.
Savitsky, E. “Acute Burn Care.” In Combat Casualty Care: Lessons Learned From OEF and OIF. Dept. of the Army, 2012.
Wagner, J. L. et al. “Extubation force depends upon angle of force application and fixation technique: a study of 7 methods.” BMC Anesthesiology, 14, 74 (2014).
Ward, C. et al. “Securing Endotracheal Tubes in Patients with Facial Burns.” American Journal of Surgery, 159, no. 3 (1990): 339-40.
Wolf, S. et al. “Comparison between civilian burns and combat burns from Operation Iraqi Freedom and Operation Enduring Freedom.” Annals of Surgery, 243, no. 6 (2006):786-95.

What is claimed is:

1) An endotracheal tube stabilization device for retention of a tube of compressible material having a predetermined, substantially uniform outside diameter, said stabilization device comprising:
a) an endotracheal tube holder having an elongated, straight generally tubular, body with an axial bore of a predetermined diameter for receiving said tube, said endotracheal tube holder having a proximal portion, an intermediate bite block and a distal portion;
b) a removable wing plate having at least two wings and a center opening, which is adapted to fit onto said endotracheal tube holder between said proximal portion and said intermediate bite block, with wings extending on the sides of said endotracheal tube holder; each of said wings has a peripheral slot; and
c) a strap assembly, which engages the wings through its peripheral slot and hold the endotracheal tube holder in place by tightening straps around the patient's head.

2) The endotracheal tube stabilization device of claim 1, wherein said axial bore has a slot along its length that is wider on the proximal portion of ETH and sized to receive an endotracheal tube from its side.

3) The endotracheal tube stabilization device of claim 1, wherein said bite block has a generally square outer shape.

4) The endotracheal tube stabilization device of claim 1, wherein said distal portion comprises:
a) an inside surface, which is lined with a grip pattern;
b) a fastener secured onto an outside of said distal portion of ETH.

5) The endotracheal tube stabilization device of claim 1, wherein said removable wing plate may freely rotate radially around endotracheal tube holder.

6) The endotracheal tube stabilization device of claim 1, wherein said straps assembly further comprise an inner lining made of a burn-compatible material.

7) The endotracheal tube stabilization device of claim 6, wherein said burn-compatible material is a nonadherent film, a fine mesh gauze, a foam, an alginate, a hydrocolloid, or a hydrogel.

8) The endotracheal tube stabilization device of claim 6, wherein said inner lining further comprises a topical antimicrobials agent.

10) A method for securing an endotracheal tube to a patient using endotracheal tube stabilization device of claim 1, comprising
a) inserting an endotracheal tube through a patient's oral cavity into upper airway;
b) orienting the proximal portion of the ETH toward the patient's oral cavity;
c) guiding the ETH onto the endotracheal tube by pressing the endotracheal tube through the slot beginning from proximal portion and into the axial bore;
d) wrapping the strap assembly around the back of the patient's head;
c) securing the strap assembly to the wing plate;
d) sliding the ETSD down the ET tube, and positioning the bite block between the patient's incisors;
e) rotating the wing plate 90 degrees, either clockwise or counter clockwise around the ET tube, allowing the rotating wing plate to close the slot on the proximal portion; and
f) tightening the adjustable fastener and engaging the endotracheal tube with the grip pattern within the distal portion of the ETH, locking the endotracheal tube in place in relation to the endotracheal tube holder.
